(19) United States
(12) Patent Application Publication
(10) Pub. No.: US 2025/0134582 A1
(43) Pub. Date: May 1, 2025

(54) CARPAL TUNNEL RELEASE SYSTEM

(71) Applicant: Arthrex, Inc., Naples, FL (US)

(72) Inventors: Ryan Kellar, Naples, FL (US); James Kitchen, Fort Myers, FL (US); Peter S. Kim, Newton, MA (US); Steven S. Shin, Los Angeles, CA (US); Sanjeev Kakar, Rochester, MN (US); Mollie Manley, Richfield, OH (US); Joshua Best, Naples, FL (US); Benjamin Siegel, Naples, FL (US); Kevin D. Martin, Delaware, OH (US); Matthew Johnson, Bonita Springs, FL (US)

(73) Assignee: Arthrex, Inc, Naples, FL (US)

(21) Appl. No.: 18/924,136

(22) Filed: Oct. 23, 2024

Related U.S. Application Data
(60) Provisional application No. 63/545,971, filed on Oct. 27, 2023.

(51) Int. Cl. A61B 18/14; A61B 90/00

(52) U.S. Cl. CPC A61B 18/1477; A61B 18/148; A61B 2018/1432; A61B 2018/1475; A61B 2090/3925

(57) ABSTRACT

A probe apparatus includes a plurality of nested sheaths including an inner sheath forming an inner sheath passage and an outer sheath forming an outer sheath passage. A cannula forms an actuator linkage that extends from a proximal linkage end portion to a distal linkage end portion. The actuator linkage extends through the inner sheath passage from the proximal end portion at a main body forming a handle portion to the distal linkage end portion and forms a lumen therethrough. A blade is in connection with the distal linkage end portion and the inner sheath. An imaging apparatus includes an imager extending through the cannula at the distal linkage end portion and captures image data in a field of view.

### CROSS-REFERENCE TO RELATED APPLICATIONS

This application claims priority under 35 U.S.C. § 119 (e) and the benefit of U.S. Provisional Application No. 63/545,971 entitled CARPAL TUNNEL RELEASE SYSTEM, filed on Oct. 27, 2023, by Ryan Kellar, et al., the entire disclosure of which is incorporated herein by reference.

### BACKGROUND

The disclosure generally relates to an apparatus and visualization system for minimally invasive surgical procedures and, more particularly, to an arthroscopic surgical device configured to access and treat narrow anatomical structures or canals. Arthroscopic surgical apparatuses and techniques provide highly specialized treatments for a variety of ailments. While providing improved results, such specialization may result in an increased number of surgical instruments to suit the requirements of various procedures as well as the preferences of surgeons. The disclosure provides for a surgical apparatus and corresponding methods to improve patient care.

### SUMMARY

The disclosure generally provides for a probe apparatus and corresponding surgical methods or techniques that may be implemented to conduct minimally invasive procedures. In various implementations, such procedures may include the identification and selective cutting or treatment of patient tissue positioned within the patient anatomy, including connective tissue, fatty tissue, and similar tissue that may be remotely accessed. In various implementations, the disclosure may provide for advanced techniques and features for a probe apparatus configured to access and treat a carpal tunnel ligament via an incision in the wrist of a patient. In general, the disclosed probe apparatus may provide for several features, including a plurality of nested sheaves, a curved access probe, improved visualization, and flexibility of operation to suit user preferences and improve patient outcomes. Though these features are described in reference to a common apparatus, the features may be implemented individually or in various combinations.

In some implementations, the probe apparatus may include a plurality of nested sheaths that may include an inner sheath, as well as an outer sheath or dilatator sheath that may be deployed independently. A lumen may extend through an inner passage within the inner sheath and may form an actuator linkage configured to adjust an incision angle of a blade as well as the position of an imaging device. The imaging device may be disposed at a distal end of the probe apparatus and form a portion of an imaging apparatus having a wired communication interface that may extend through a cannula formed by the lumen of the actuator linkage. As provided in further detail in the various examples that follow, the operation of the probe apparatus may provide for improved access and visualization of a target region of the patient anatomy to improve patient outcomes from surgical procedures.

In some implementations, the probe apparatus may be utilized in combination with a handle adapter that may convert a handle interface and corresponding interaction of the surgical probe from a fist-grip or cylindrical-grip to a pistol-grip. The handle adapter may include an actuator assembly including a trigger adapter that may be configured to engage an actuation input of the probe otherwise engaged by a user in the fist-grip configuration. The handle adapter may be implemented via a mounting interface that may be selectively deployed with the probe apparatus during various stages of a surgical procedure. Accordingly, the disclosure may provide for the probe apparatus to be implemented to suit a variety of user preferences based on these features and various detailed exemplary configurations discussed in the following detailed description.

These and other features, objects and advantages of the present disclosure will become apparent upon reading the following description thereof together with reference to the accompanying drawings.

### BRIEF DESCRIPTION OF THE DRAWINGS

FIG. 1 is a perspective view of a probe apparatus demonstrating an exemplary operation of a distal blade;

FIG. 2 is a partially exploded, perspective view of a probe apparatus demonstrating an outer sheath separated from an inner sheath and a handle portion;

FIG. 3 is an exploded assembly view of a probe apparatus;

FIG. 4 is a side profile view demonstrating a probe apparatus in an undeployed configuration and a deployed configuration;

FIG. 5A is a side profile view of a probe apparatus demonstrating a field of view of a camera in a first rotational position;

FIG. 5B is a side profile view of a probe apparatus demonstrating a field of view of a camera in a second rotational position;

FIG. 6A is a detailed, partially exploded assembly view of an actuator assembly of the probe apparatus;

FIG. 6B is a detailed perspective view demonstrating a longitudinal actuator of the actuation assembly demonstrated in FIG. 6A;

FIG. 7A is a side profile view demonstrating an implementation of a probe apparatus having an angle camera head;

FIG. 7B is a side profile view demonstrating an implementation of a probe apparatus having a curved engagement profile;

FIG. 8A is a partially exploded assembly view of a probe apparatus being loaded into a handle adapter;

FIG. 8B is an assembly view of the probe apparatus being mounted to the handle adapter being demonstrated in FIG. 8A;

FIG. 8C is an assembly view of the probe apparatus of FIGS. 8A and 8B mounted in the handle adapter demonstrating an actuation of a surgical blade;

FIG. 8D is an assembly view of the probe apparatus and the handle adapter of FIGS. 8A, 8B, an 8C demonstrating an unloading procedure;

FIG. 9A is an exemplary sample of image data demonstrating a field of view corrected to demonstrate a local environment consistently relative to gravity during a rotation of an imager;

FIG. 9B is an exemplary sample of image data demonstrating a field of view corrected to demonstrate a local environment consistently relative to gravity during a rotation of an imager; and

FIG. 10 is a block diagram demonstrating an imaging system and controller for an imaging apparatus in accordance with the disclosure.

### DETAILED DESCRIPTION

In the following description, reference is made to the accompanying drawings, which show specific implementations that may be practiced. Wherever possible, the same reference numbers will be used throughout the drawings to refer to the same or similar parts or features. It is to be understood that other implementations may be utilized and structural and functional changes may be made without departing from the scope of this disclosure.

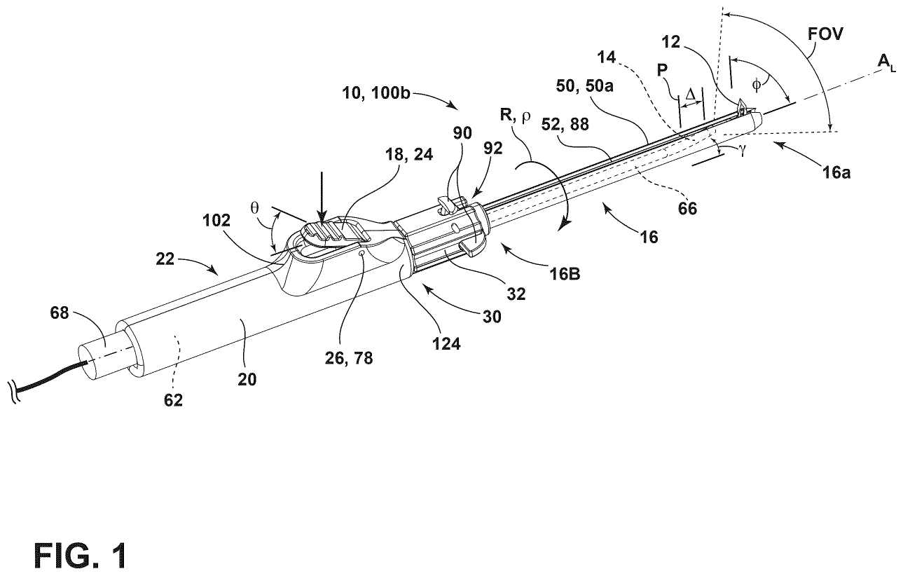

As generally demonstrated in FIGS. 1-8, the disclosure provides for a variety of features and accessories that may be implemented alone or in combination to improve the operation of a probe apparatus 10 designed to effectuate minimally invasive surgical procedures. In general, the probe apparatus 10 may include a distal blade 12 and imager 14 that may be selectively deployed from a distal end portion 16a of a probe body 16. In operation, the blade 12 and imager 14 may be actuated in response to an input, which may be in the form of an angular or positional adjustment of an actuation input 18 positioned on a handle portion 20 of a main body 22 of the probe apparatus 10. As demonstrated in various examples, the actuation input 18 may correspond to an actuation lever 24 that may be pivotally connected to the main body 22 via a pin 26 or pivot assembly that may correspond to a fulcrum of the actuation lever 24. As shown in FIG. 1, an adjustment of a lever angle θ of the actuation lever 24 may result in a change in a deployment angle ϕ of the blade 12. In addition to the adjustment of the deployment angle ϕ of the blade 12, adjustment of the lever angle θ may result in a longitudinal displacement Δ of a position P of the imager 14. In this way, the operation of an actuation assembly 30 controlled by the position of the actuation input 18 may adjust the field of view (FOV) to focus on the changing deployment angle ϕ of the blade 12.

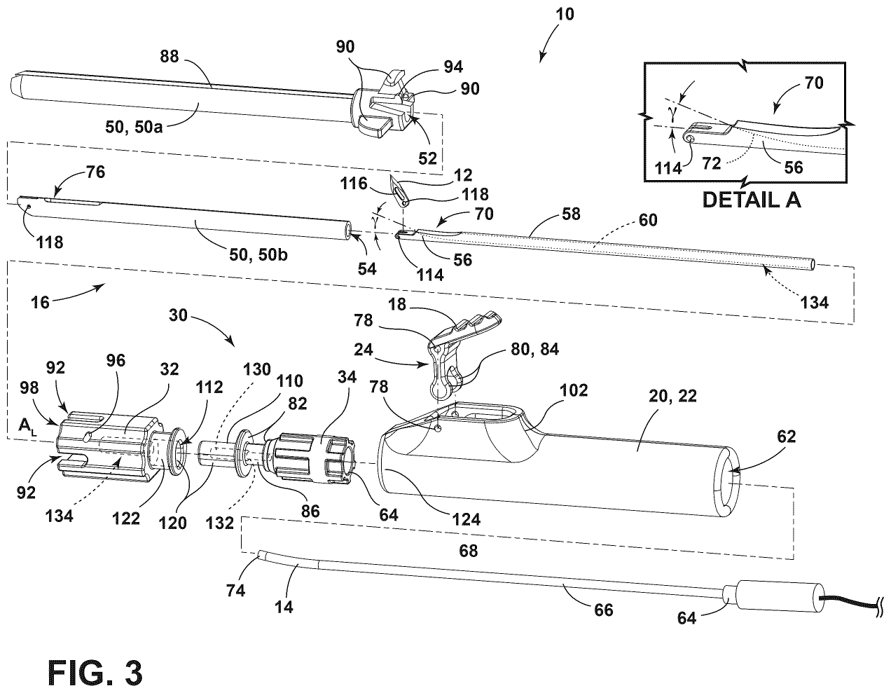

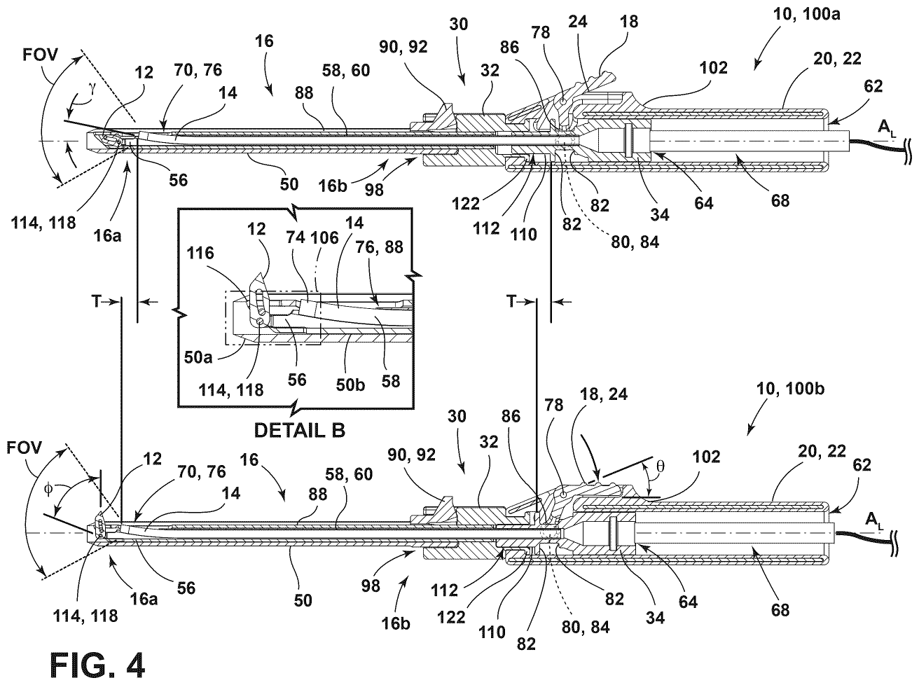

In some implementations, the disclosure may further provide for the probe body 16 to rotate, as denoted by the arrow R, about a longitudinal axis A_{L} relative to the main body 22 including the handle portion 20. As shown, a probe rotation R may be facilitated by engagement of a rotational hub 32 in connection with the main body 22 of the probe apparatus 10. As further discussed in reference to FIGS. 4-6, the operation of the actuation assembly 30 in combination with the probe rotation R, may be effectuated by an engagement between the rotational hub 32 to the main body 22. Additionally, the rotational hub 32 may be connected to a longitudinal actuator 34 (see FIGS. 3, 4, and 6) that may provide for an adjustment of a position and/or orientation of the blade 12 and the imager 14 based on the lever angle θ of the actuator lever 24. In this configuration, the blade 12 and imager 14 may be selectively oriented or rotated R about the longitudinal axis A_{L} at a probe angle ρ and deployed at the deployment angle ϕ.

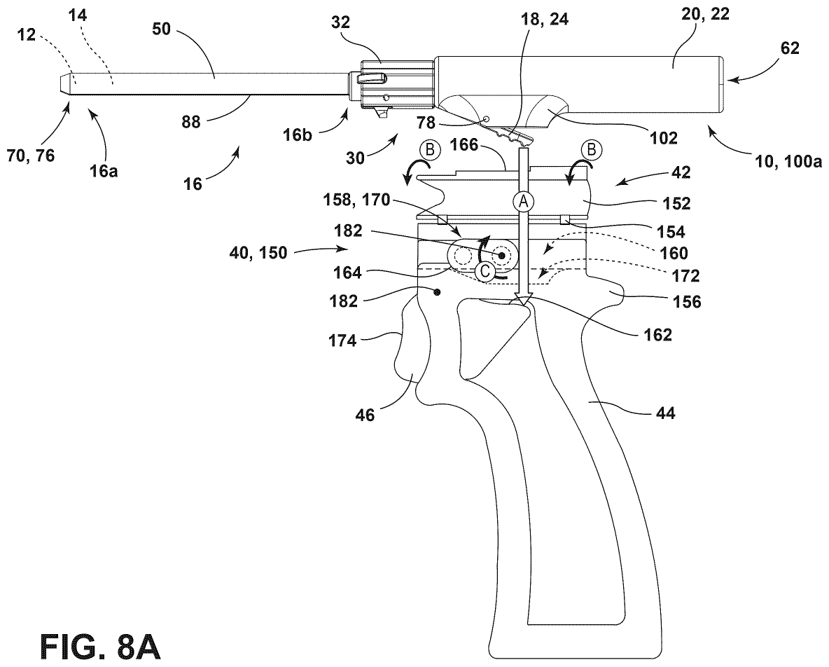

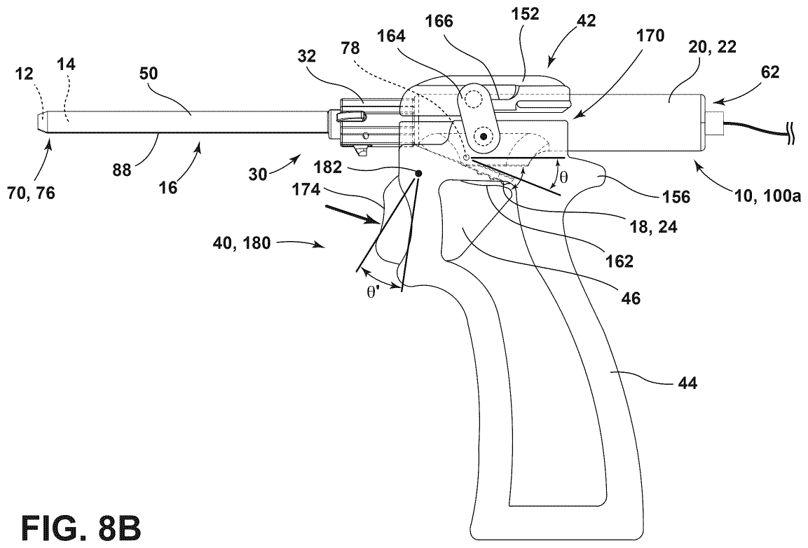

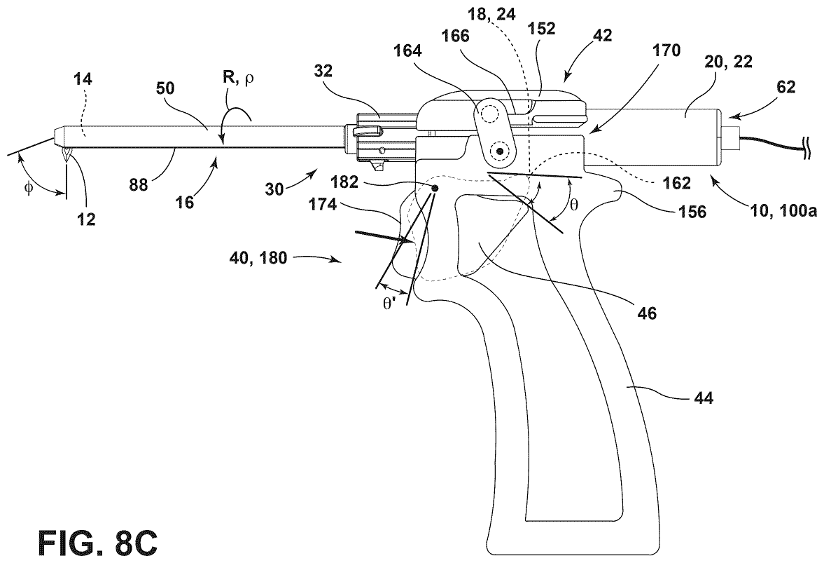

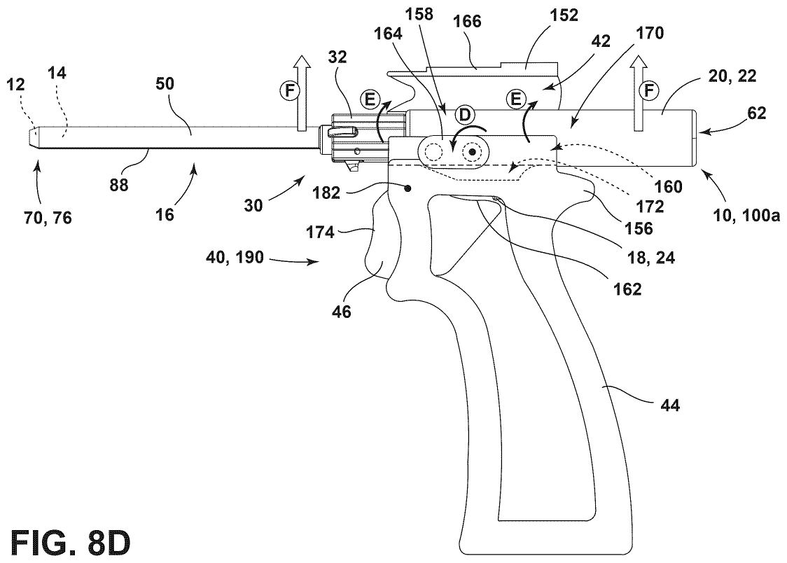

In addition to the various operating features and functions provided by the probe apparatus 10, the disclosure may further provide for a handle adapter 40 as later discussed in reference to FIGS. 8A-8D. In various implementations, the handle adapter 40 may provide for a closure 42 or connection interface that may be configured to engage and retain the probe apparatus 10 in connection with a handle body 44 of the adapter 40. In this configuration, a cylindrical or fist-grip associated with the probe apparatus 10 may be adapted to a pistol-grip by selectively engaging the actuation lever 24 via a trigger adapter 46. In such implementations, the probe apparatus 10 may be implemented to flexibly suit a variety of preferred handling techniques that may support operation between the fist-grip or the pistol-grip while also supporting a right or left handedness of a user. Accordingly, the disclosure may provide for a variety of features that may be implemented, alone or in various combinations, to improve the operation of the probe apparatus 10.

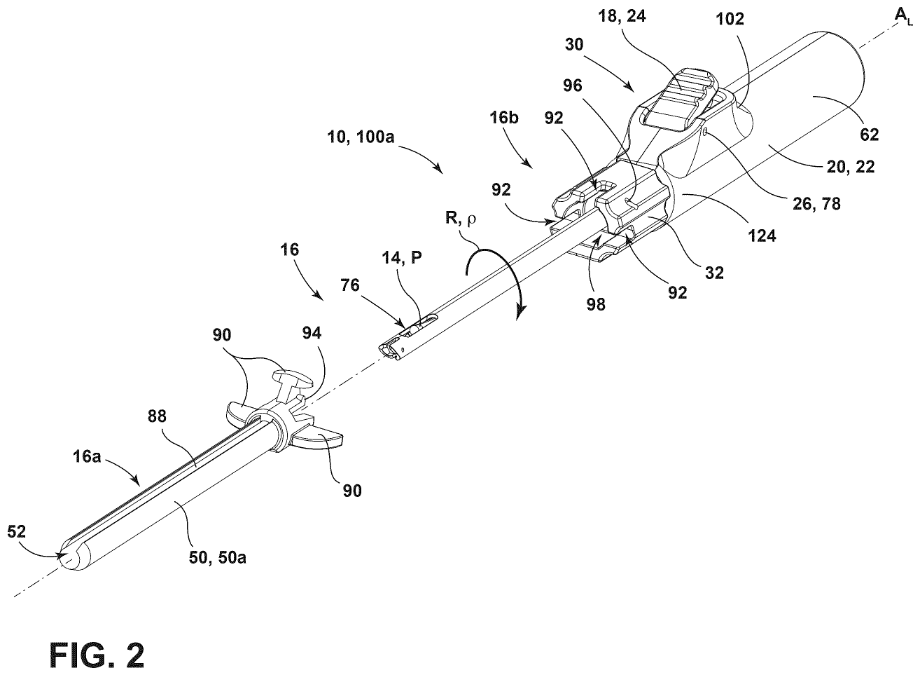

Referring now to FIGS. 2-3, in various implementations, the probe apparatus 10 may comprise a plurality of sheaths 50 that may be assembled in a nested configuration. As shown in FIG. 2, the plurality of sheaths 50 may include an outer sheath 50a or dilator sheath forming an outer sheath passage 52 through which an inner sheath 50b may be selectively inserted to access a target region proximate to the distal end portion 16a of the probe 16. The nested configuration of the plurality of sheaths 50 may allow a user to independently deploy the outer sheath 50a and selectively deploy the inner sheath 50b. The successive insertion of the outer sheath 50a and the inner sheath 50b may provide for the outer sheath 50a to serve as a dilator or initial positioning probe prior to deployment and insertion of the inner sheath 50b containing the blade 12. Additionally, the selective deployment of the inner sheath 50b may allow the functional components of the probe apparatus 10, including the blade 12 and the imager 14, to be rapidly removed and reinserted into the patient anatomy without interrupting the corresponding procedure.

As demonstrated in FIG. 3, the inner sheath 50b may form an inner sheath passage 54 through which an actuator linkage 56 may be positioned along a length of the probe 16 to adjust the deployment angle ϕ and the camera position P. The actuator linkage 56 may be implemented in the form of a narrow tubular structure or cannula 58 forming an interior lumen 60. In the assembled configuration as demonstrated in FIG. 2, the cannula 58 may extend from a proximal end portion 16b of the probe body 16 to the distal end portion 16a. From the proximal end portion 16b, the cannula 58 forming the actuator linkage 56 may extend into a receiving cavity 62 of the main body 22. In this configuration, the actuator linkage 56 may engage the longitudinal actuator 34 and extend to the imager 14 and the blade 12 to effectuate the longitudinal displacement Δ and deployment angle ϕ.

As demonstrated in FIG. 3, an elongated or wire-like body 66 of an imager apparatus 68 comprising the imager 14 may be inserted into the receiving cavity 62 and pass through a portion of the actuation assembly 30 and the lumen 60 to extend along a length of the probe 16 to the distal end portion 16a. A distal end of the actuator linkage 56 formed by the cannula 58 may form a sloped or inclined opening 70 that may be angled at a projection angle or camera angle γ relative to the longitudinal axis A_{L} of the probe body 16. As best demonstrated in DETAIL A of FIG. 3, the inclined opening 70 may extend through a side wall of the cannula 58 and may include a ramp 72 or inclined feature upon which a head 74 or imaging receiver of the imager 14 may rest to align the field of view FOV of the imager 14 along the camera angle γ. As best illustrated in FIG. 2, the head 74 of the imager 14 may protrude from the inclined opening 70, through the cannula 58, and into an imaging slot 76 formed through a distal end portion of the inner sheath 50b. In this configuration, the field of view of the imager 14 may be projected out through the imaging slot 76 with the cannula 58 and imaging apparatus 68 deployed in the inner sheath 50b. In addition to the imaging receiver, the head 74 of the imager 14 may further include one or more lighting elements or arrays configured to illuminate the field of view FOV. The path of the lumen 60 along the ramp 72 may be aligned with a cutting edge of the blade 12 at the camera angle γ, such that the blade 12 is centrally captured and illuminated in the field of view FOV.

In addition to the deployment of the blade 12, the displacement Δ of the head 74 of the imager 14 responsive to the translation of the actuator linkage 56 may adjust the proximity of the field of view FOV and illumination responsive to the deployment angle ϕ of the blade 12. In this way, the deployment of the blade 12 may be accompanied by a corresponding translation of the field of view FOV toward the blade 12 improving the detail and illumination focused on the blade 12 and effectively cropping the environment about the perimeter of the field of view FOV. As further discussed and demonstrated in reference to FIGS. 4-6, the probe rotation R and corresponding probe angle ρ of the probe body 16 may result in the camera angle γ and corresponding alignment of the deployment angle ϕ of the blade 12 to be selectively adjusted about the longitudinal axis A_{L}. Such operation may provide for flexible deployment and positioning of the main body 22 and handle portion 20 relative to the probe body 16.

Before continuing to discuss other features of the actuation assembly 30, FIG. 4 demonstrates a projected view of the actuation lever 24 including a receiving aperture 78 that may receive the pivot pin 26 and interconnect the actuation lever 24 to the main body 22. Opposite the actuation input 18 of the actuation lever 24, at least one engagement arm 80 may extend opposite the actuation input 18. In an assembled configuration as demonstrated in FIG. 4, the at least one engagement arm 80 may extend into the receiving cavity 62 of the main body 22 and be positioned between a pair of opposing actuation surfaces 82 longitudinally spaced along the length of the longitudinal actuator 34. As shown, the at least one engagement arm 80 may correspond to a plurality of engagement arms or prongs that may form a wishbone-shaped actuator 84 that extends along opposite sides of a support column 86 formed by the longitudinal actuator 34 between the opposing actuation surfaces 82. In operation, the adjustment of the lever angle θ may cause the at least one engagement arm 80 to apply force to either of the opposing actuation surfaces 82 resulting in a proximal or distal translation T of the longitudinal actuator 34 relative to the main body 22 and the rotational hub 32 as demonstrated in FIG. 4. In this configuration, the actuation assembly 30 may provide for the deployment and operation of the inner sheath 50b and actuation assembly 30 for selective engagement with the outer sheath 50a.

As previously discussed in reference to FIG. 2, the inner sheath 50b in connection with the probe apparatus 10 may be selectively inserted into the outer sheath passage 52 of the outer sheath 50a. In this configuration, the probe apparatus 10 may be selectively inserted or removed from the outer sheath 50a when the outer sheath 50a is deployed within the anatomy of a patient. During the insertion of the inner sheath 50b into the outer sheath passage 52 of the outer sheath 50a, the field of view FOV of the imager 14 may be directed upward at the camera angle γ toward an elongated slot 88 formed longitudinally along a length of the outer sheath 50a. For example, the field of view FOV of the imager 14 may be presented to a user on a display screen providing visualization of the anatomy adjacent to the elongated slot 88 extending along the length of the outer sheath 50a during the insertion of the inner sheath 50b. In this way, the selective deployment of the probe apparatus 10 and the inner sheath 50b may allow a user to survey the patient anatomy extending along the length of the outer sheath 50a during an initial deployment or successive deployments of the probe apparatus 10 within the patient cavity. In some cases, the outer sheath 50a may correspond to a transparent material (e.g., transparent polymeric material), such that the field of view FOV of the imager 14 may capture image data demonstrating the patient anatomy about the length of the outer sheath 50a as well as through the elongated slot 88. Such configurations may provide for improved visibility, allowing users to survey the patient anatomy on all sides of the outer sheath passage 52 while inserting and withdrawing the inner sheath 50b of the probe apparatus 10 from the outer sheath 50a.

As best shown in FIGS. 2 and 3, the nested engagement between the outer sheath 50a and the inner sheath 50b may be aligned by at least one alignment tab 90 and corresponding alignment aperture 92 of the outer sheath 50a and the rotational hub 32. As shown, the at least one alignment tab 90 may correspond to a plurality of alignment tabs 90 that may be received by corresponding rotationally distributed alignment apertures 92 positioned about the rotational hub 32. In operation, the outer sheath 50a may be rotationally coupled to the rotational hub 32 via the engagement of the alignment tabs 90 with the alignment apertures 92. Additionally, the outer sheath 50a and rotational hub 32 may include one or more positioning detents 94 configured to longitudinally couple the outer sheath 50a to the rotational hub 32. In the example shown, the positioning detent 94 may be positioned on a proximal end portion of a central alignment tab 90 of the outer sheath 50a. The positioning detent 94 may be received by a complementary indentation 96 formed within a distal opening 98 of the rotational hub 32. In this configuration, the outer sheath passage 52 of the outer sheath 50a may be slidably engaged by the inner sheath 50b from a proximal end portion 16b to a distal end portion 16a of the probe body 16. Additionally, the outer sheath 50a may be rotationally and longitudinally coupled to the rotational hub 32 via an engagement between the alignment tabs 90 with the alignment apertures 92 and the positioning detents 94 with the receiving indentations 96.

Referring again to FIG. 4, the probe apparatus 10 is demonstrated in an undeployed configuration 100a and a deployed configuration 100b, illustrating the longitudinal displacement Δ of the imager 14 and the actuator linkage 56 in response to the engagement of the actuation lever 24 with the opposing actuation surfaces 82 of the longitudinal actuator 34. In the undeployed configuration 100a, the lever angle θ is extended away from a protruding engagement surface 102 formed along the handle portion 20 about the actuation lever 24. In this configuration, the blade 12 may be undeployed and positioned within an access perimeter 106 defining an access envelope formed by an exterior wall of the outer sheath 50a. In operation, the actuation input 18 of the actuation lever 24 may be pressed toward the main body 22, thereby causing the at least one engagement arm 80 to apply a distal force to the corresponding actuation surface 82 of the longitudinal actuator 34. The longitudinal force applied to the longitudinal actuator 34 may cause a distal protrusion 110 of the longitudinal actuator 34 to extend into an actuation aperture 112 or longitudinal aperture formed through a proximal end portion of the rotational hub 32. Additionally, the translation of the distal protrusion 110 into the actuation aperture 112 may result in the connected actuator linkage 56 to slide within the inner sheath passage 54. The translation of the actuator linkage 56 in the inner sheath 50b may adjust the deployment angle ϕ of the blade 12 as well as the imager position P of the imager 14 over the displacement distance A. In this way, the adjustment of the lever angle θ of the actuation lever 24 may cause the position of the imager 14 to change responsive to or contemporaneous with the deployment of the blade 12 in the deployed configuration 100b.

Though not shown, a spring or spring mechanism (e.g., a coil spring, torsion spring, etc.) may be positioned between the longitudinal actuator 34 and the rotational hub 32. In operation, the spring may oppose the distal translation T of the longitudinal actuator 34 and bias the actuation lever 24 away from the main body 22 in the undeployed configuration 100a. In response to the application of force to the actuation input 18, a spring force may oppose the change in the lever angle θ. In this configuration, the spring force may return the probe apparatus 10 from the deployed configuration 100b to the undeployed configuration 100a as a result of the release of the force applied to the actuation input 18 and the extension of the spring from a compressed condition.

As best demonstrated in DETAIL B of FIG. 4, the blade 12 may be connected to a distal actuator coupling 114 of the actuator linkage 56 at a proximal end of the blade 12. Additionally, the blade 12 may comprise a curved slot 116 engaged by a distal sheath coupling 118 formed at a distal end portion of the inner sheath 50b. In this configuration, the translation or displacement Δ of the actuator linkage 56 may cause the distal actuator coupling 114 to drive the blade 12 relative to the distal sheath coupling 118, resulting in a combined translation and rotation of the blade 12 along the curved slot 116. In the example shown, the distal actuator coupling 114 and the distal sheath coupling 118 are implemented as pin connections, allowing the blade 12 to translate and rotate along the curved slot 116 to effectuate the deployment along the deployment angle ϕ. However, it shall be understood that other connections may be implemented.

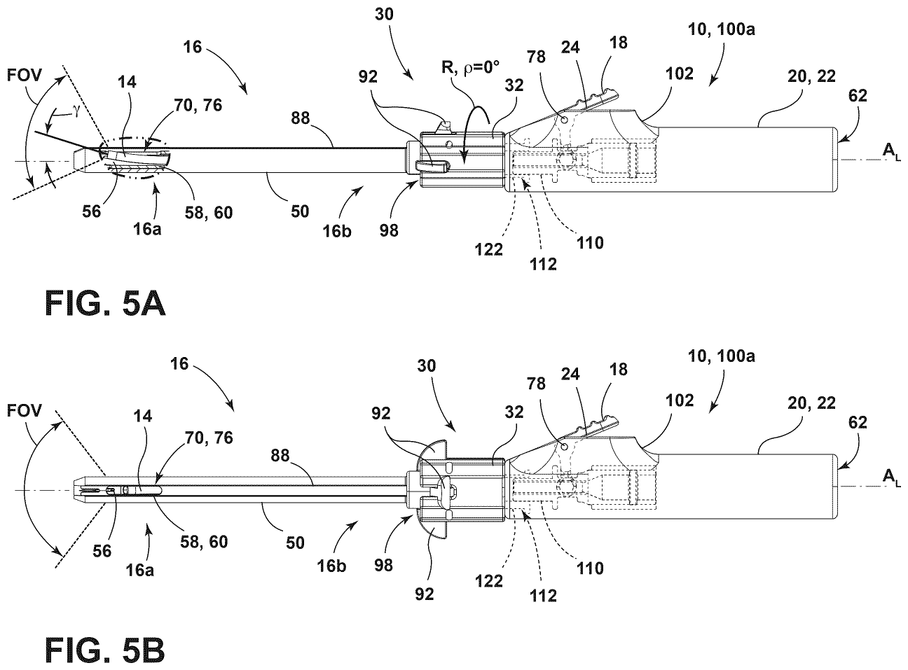

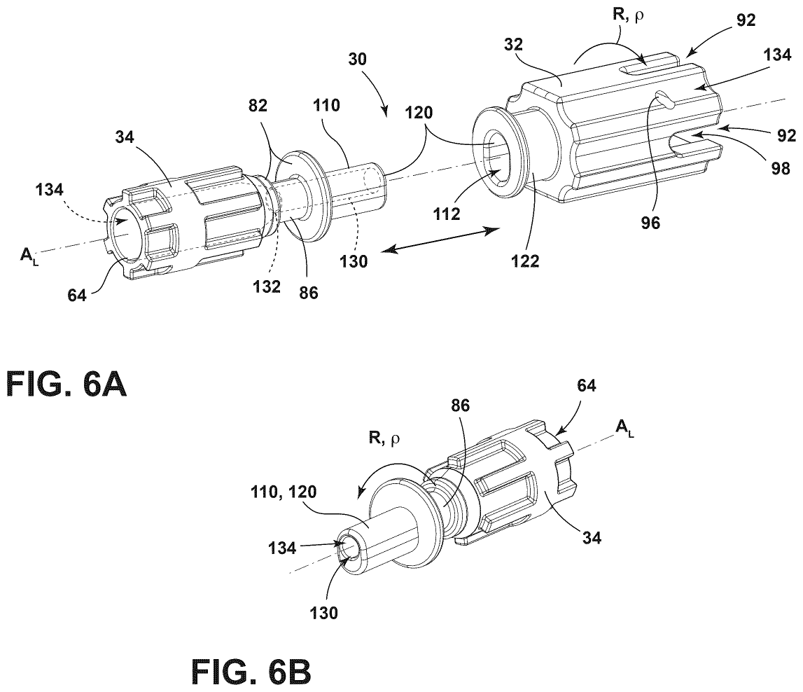

Referring now to FIGS. 5 and 6, the operation of the actuation assembly 30 is further described in reference to the probe rotation R and displacement Δ of the actuator linkage 56 for clarity. As previously discussed, the configuration of the actuation assembly 30 may provide for a user to rotate the rotational hub 32 relative to the main body 22 of the probe apparatus 10. As demonstrated in FIGS. 5A and 5B, the probe rotation R is demonstrated at a neutral probe angle γ=0 and a probe angle γ=90°, respectively. In operation, the rotational hub 32 may provide for a connection to the main body 22 of the probe apparatus 10 to allow the underlying and interconnected longitudinal actuator 34, inner sheath 50b, the actuator linkage 56, and the cannula 58 to rotate relative to the main body 22 and handle portion 20 as demonstrated by the probe rotation R. In this configuration, the actuation assembly 30 may rotate relative to the main body 22 of the probe apparatus 10 via the rotational hub 32. As demonstrated by comparing FIGS. 5A and 5B, the camera angle γ and orientation of the elongated slot 88 formed through the outer sheath 50a or dilator sheath may rotate about the longitudinal axis A_{L} in conjunction with the rotation R of the rotational hub 32.

As demonstrated in FIGS. 6A and 6B, the displacement Δ or travel of the actuator linkage 56 and the cannula 58 may be effectuated by the sliding engagement between the longitudinal actuator 34 and the rotational hub 32. In operation, the distal protrusion 110 of the longitudinal actuator 34 may engage a depth of the actuation aperture 112 formed in a proximal portion of the rotational hub 32. In this configuration, changes in the lever angle θ of the actuation lever 24 may result in the at least one engagement arm 80 translating T the longitudinal actuator 34 along the longitudinal axis A_{L} by engaging the opposing actuation surfaces 82 along the support column 86. As a result, the distal protrusion 110 may engage the actuation aperture 112 via a piston-like engagement. As shown, the distal protrusion 110 and/or the actuation aperture 112 or longitudinal aperture may form a profile shape 120 that may be noncircular, irregular, or otherwise shaped to inhibit the longitudinal actuator 34 from rotating relative to the rotational hub 32. In this way, the actuation assembly 30 may be free to allow the longitudinal actuator 34 to translate along the longitudinal axis A_{L} while rotationally coupling the longitudinal actuator 34 to the rotational hub 32 and the main body 22.

Still referring to FIGS. 6A and 6B, the proximal end portion of the rotational hub 32 may form a neck or rotational protrusion 122 that may be enclosed by a collar 124 at a distal end portion of the main body 22. The engagement of the collar 124 about the rotational protrusion 122 is best demonstrated by the hidden lines shown in FIGS. 5A and 5B. As shown, a circular perimeter wall of the rotational protrusion 122 may rotate freely within the collar 124 about the longitudinal axis A_{L}. As illustrated by comparing FIGS. 5A and 5B, the cross-sectional profile of the rotational protrusion 122 is radially consistent about the longitudinal axis A_{L} allowing the rotational hub 32 to rotate within the collar 124 relative to the main body 22 and the handle portion 20. In various implementations, the main body 22 may be manufactured in a two-piece assembly that divides the collar 124 into a plurality of parts that can be enclosed about the rotational protrusion 122 during assembly. Though specific details of the exemplary probe apparatus 10 are discussed, it shall be understood that variations in the manufacturing and design may also be supported by the teachings of the disclosure.

Referring still to FIGS. 6A and 6B, the longitudinal actuator 34 may form a linkage-receiving aperture 130 that may be configured to receive and retain a proximal end portion of the cannula 58 forming the actuator linkage 56. As shown in FIG. 6A, a proximal end portion of the linkage-receiving aperture 130 may be partially enclosed, such that the proximal end portion of the cannula 58 may engage an interior shelf 132 forming a positive stop receiving a predetermined length of the cannula 58. As additionally shown in FIG. 6A, the longitudinal actuator 34 and rotational hub 32 may combine to form an elongated interior passage 134 that may extend from a proximal end portion of the longitudinal actuator 34, through the distal protrusion 110 and the actuation aperture 112 and out through the distal opening 98 of the rotational hub 32. The elongated interior passage 134 may extend through the actuation assembly 30 within the lumen 60 formed by the cannula 58 within the inner sheath passage 54 of the inner sheath 50b. In this configuration, the inner sheath 50b may extend into and be retained within the elongated interior passage 134 formed within the rotational hub 32. The cannula 58 and corresponding actuator linkage 56 may extend through the inner sheath passage 54 from the actuator linkage-receiving aperture 130 formed by the distal protrusion 110 and out to the distal end portion of the actuator linkage 56 forming the distal actuator coupling 114. In this configuration, the telescoping engagement between the longitudinal actuator 34 and the rotational hub 32 may result in the actuator linkage 56 and the cannula 58 translating longitudinally along the longitudinal displacement A. As a result, the imager 14 and the blade 12 may be actuated in conjunction as demonstrated in FIG. 4.

Finally, as best demonstrated in FIG. 6B, the linkage-receiving aperture 130 formed within the distal protrusion 110 of the longitudinal actuator 34 may be aligned with the longitudinal axis A_{L} of the rotational hub 32. In this configuration, the elongated or irregular profile shape 120 of the distal protrusion 110 may be offset from the linkage-receiving aperture 130. The noncircular or offset profile shape 120 of the distal protrusion 110 may ensure that the rotational hub 32 is rotationally coupled about the longitudinal axis A_{L} to the longitudinal actuator 34. In this configuration, the orientation of the field of view FOV of the imager 14 may be rotated in combination with the coupling interface 64 and the imager apparatus 68 extending through the main body 22 for ease of operation.

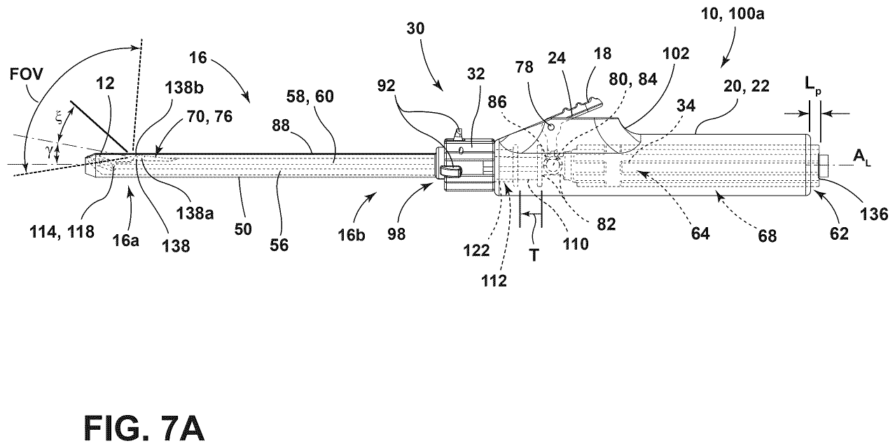

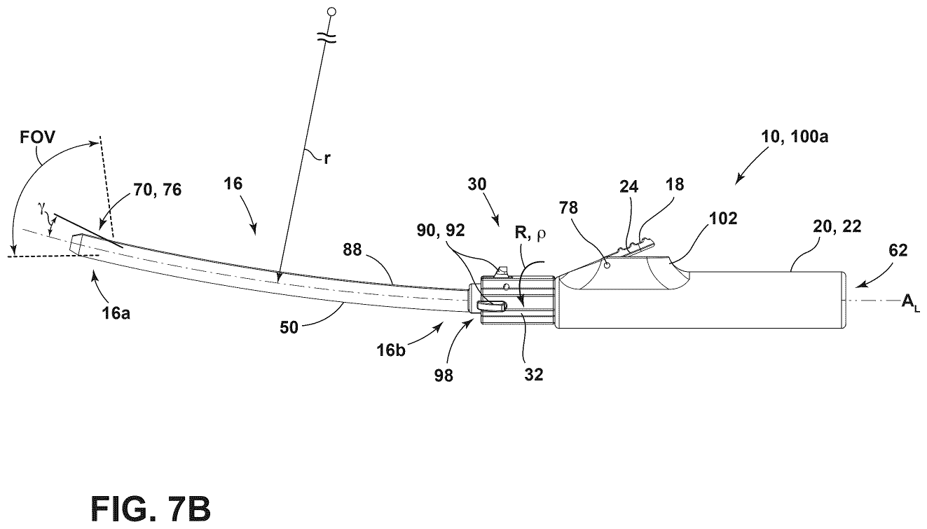

Referring now to FIGS. 7A and 7B, in various implementations, one or more components of the probe apparatus 10 may vary depending on the application or user preference. As demonstrated in FIG. 7A, the body of the longitudinal actuator 34 may extend proximally from the handle portion 20 of the main body 22. In this configuration the proximal end portion of the longitudinal actuator 34 may protrude from the handle portion 20 and form an adapter protrusion 136 that may traverse the receiving cavity 62 to vary in a protrusion length Lₚ extending from the handle portion 20 in response to the position of the actuation lever 24 and the corresponding position of the longitudinal actuator 34 along the length of the main body 22. In this configuration, the coupling interface 64 of the imager apparatus 68 may protrude from the receiving cavity 62 over a variable protrusion length Lₚ in response to the lever angle θ and the translation T of the longitudinal actuator 34. For additional information regarding the translation T of the longitudinal actuator 34, refer back to FIG. 4.

In addition to the longitudinal actuator 34, FIG. 7A further demonstrates an example of the imager apparatus 68 including an imager 138 with angled field of view FOV. As previously discussed in reference to FIG. 4, the field of view FOV of the imager 14 may be directed upward at the camera angle γ toward the elongated slot 88 formed longitudinally along a length of the outer sheath 50a. Additionally, the head 74 of the imager 14 may extend and retract along a path of the lumen 60 along the ramp 72. As illustrated in FIG. 7A, the head 74 of the imager 138 may be angled relative to the length of the imager body 138a. For example, imager 138 forms an angled distal face 138b or otherwise angled distal optics that may direct the field of view FOV at a focal angle ξ. In this configuration, the distal face 138b may orient the field of view FOV of the imager 138 angled relative to the body 138a. In this configuration, the field of view FOV may be angled relative to the imager body 138a supported by the actuator linkage 56. In this way, the field of view FOV of the imager 138 may be directed through the imager slot 76. In this way, the imager 138 may be directed at an increased camera angle γ or the angle of the ramp 72 may be decreased or negated while still capturing image data in the field of view FOV directed at the focal angle ξ. In this way, the probe apparatus 10 may provide for the capture of image data through imager slot 76 with the imager 14 inclined along the ramp 72, with the imager 138 including the focal angle ξ implemented without the ramp 72, or with the imager 138 including the focal angle ξ and further angled along the ramp 72 at the camera angle v.

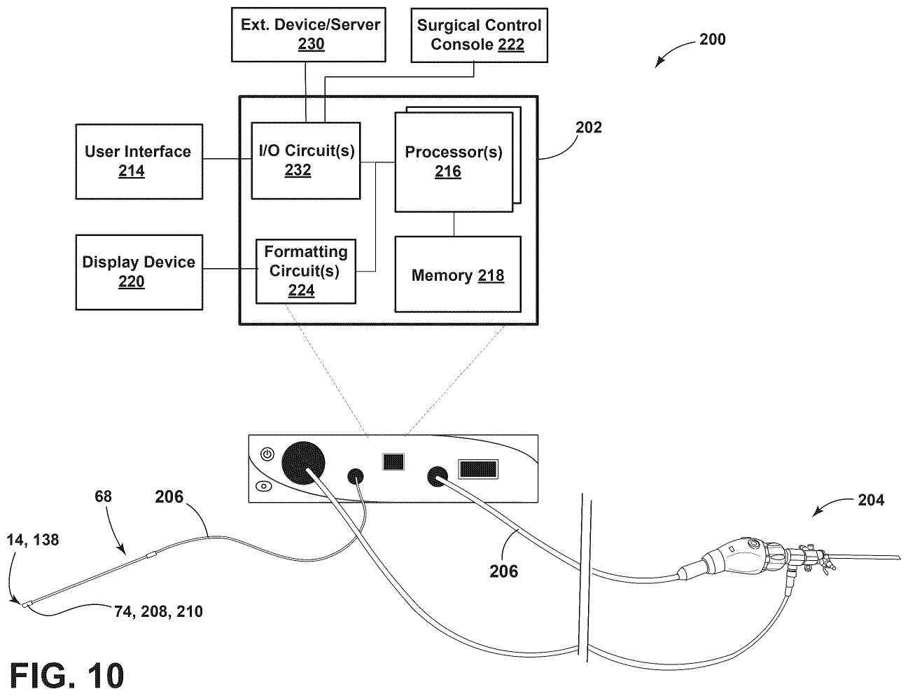

In various implementations, the imagers 14, 138 may correspond to a chip-on-tip or chip-type camera design configured to capture and communicate image data presenting a scene within the field of view FOV of an imaging lens housed in the imager body 138a. The image data may be communicated to an imaging controller (see FIG. 10) via a conductive connector. In various implementations, the imager apparatus 68 may further include one or more light sources, which may be housed in the imager body 138a to project light emissions from the angled distal face 138b. The light source(s) may correspond to light emitting diodes (LED) that may output light emissions to illuminate a scene in the field of view FOV of the imagers 14, 138. The light output from the light source(s) may correspond to one or more wavelengths of light that may include a visible light spectrum and/or other spectrums of light including a near infrared or infrared light spectrum. In an exemplary embodiment, the light source(s) may be configured to output a substantially white light or color tunable lighting emission to provide adjustable illumination in the field of view FOV.

Referring now to FIG. 7B, the probe body 16 may extend from the rotational hub 32 along a curved engagement profile 140. In some cases, the curved engagement profile 140 may correspond to a gradual radial curve having a radius that may vary between approximately 50 cm and 1000 cm. For example, the curved engagement profile 140 may extend along all or a portion of the probe body 16 between the proximal end 16b and the distal end 16a and may have a radius greater than 50 cm, 100 cm, 150 cm, 200 cm, 500 cm, 1000 cm, or any radial curve or combination of curves from approximately 50 cm to 1000 cm. While the curved engagement profile 140 is demonstrated as a gradual curve in the example of FIG. 7B, it shall be understood that the curved engagement profile 140 may vary in curvature along a length of the probe body 16.

To implement the curved probe body 16 demonstrated having the curved engagement profile 140, the plurality of sheaths 50, the actuator linkage 56, the cannula 58, and the elongated, wire-like body 66 of the imager apparatus 68 may be formed or otherwise manufactured with flexible materials that may be bent along the curved engagement profile 140. For example, one or more of the plurality of sheaths 50 and the cannula 58 may be formed by flexible metallic and/or polymeric material that may provide stability for insertion and support of the actuation of the blade 12 while also traversing or bending along the curved engagement profile 140 from the proximal end portion 16b to the distal end portion 16a. As further demonstrated in FIG. 7B, the camera angle γ may be directed away from or be oriented relative to the curved engagement profile 140 located at the distal end portion 16a of the probe body 16. In such implementations, the camera angle γ may be defined in reference to the distal end portion 16a of the curved engagement profile 140 rather than the longitudinal axis A_{L} of the main body 22 as depicted in many of the exemplary figures herein.

Referring now to FIGS. 8A-8D, the handle adapter 40 is discussed in further detail in reference to the operation of the actuation assembly 30 of the probe apparatus 10. Each of FIGS. 8A, 8B, 8C, and 8D demonstrate sequential operating configurations and steps for implementing the handle adapter 40 with the probe apparatus 10. Beginning in FIG. 8A, the handle adapter 40 is demonstrated in a loading configuration 150. In this configuration, a cover bracket 152 of the closure 42 is rotationally opened about a hinge assembly 154 connecting the cover bracket 152 to an adapter body 156 of the handle adapter 40. In the loading configuration, the main body 22, including the handle portion 20 of the probe apparatus 10, may be loaded into a receiving opening 158 formed between a recess 160 of the adapter body 156 and the cover bracket 152. To mount the probe apparatus 10 to the handle adapter 40, the main body 22 of the probe apparatus 10 may be inserted within the recess 160 of the adapter body 156. Once mounted, the actuation lever 24 is aligned with an engagement surface 162 of the trigger adapter 46.

With the probe apparatus 10 positioned within the recess 160 via the receiving opening 158, the cover bracket 152 may be rotated about the hinge assembly 154, thereby securing the main body 22 of the probe apparatus 10 to the handle adapter 40. In various implementations, the cover bracket 152 may be secured to the adapter body 156 via a latch 164. The latch 164 may be rotationally coupled to the adapter body 156 and rotationally engage a locking protrusion 166 of the cover bracket 152. In this configuration, the main body 22 of the probe apparatus 10 may be secured within the recess 160 of the handle adapter 40. As demonstrated, the latch 164 and locking protrusion 166 may correspond to a sash-lock assembly, however, it will be understood that various locking mechanisms may be implemented to secure the cover bracket 152 to the adapter body 156. As demonstrated in FIG. 8A, a loading sequence of the probe apparatus 10 may include sequential steps as follows:
(A) loading main body 22 of probe apparatus 10 into recess 160 of adapter body 156;
(B) rotating cover bracket 152 about hinge assembly 154 enclosing main body 22 within the closure 42; and
(C) rotating the latch 164 to engage the locking protrusion 166.

Following the sequential loading steps, the main body 22 of the probe apparatus 10 may be securely loaded into the recess 160 and secured to the handle adapter 40.

As shown in the various exemplary figures of the probe apparatus 10, the main body 22 may be implemented having an elongated profile shape 170. Accordingly, the recess 160 formed in the adapter body 156 may form a complementary receiving trough or channel that may cradle or support the main body 22 in connection with the handle adapter 40. In some instances, the recess 160 may include an alignment cavity 172 configured to receive the protruding surface 102 of the handle portion 20. As previously discussed, the protruding surface 102 of the handle portion 20 may extend from the main body 22 about or proximal to the actuation lever 24. In this configuration, the alignment cavity 172 may align a length of the probe apparatus 10 within the recess 160, such that the actuation input 18 of the actuation lever 24 is aligned with the engagement surface 162 of the trigger adapter 46. Once mounted to the handle adapter 40, the actuation of the trigger adapter 46 at an input surface 174 may cause the engagement surface 162 to depress the actuation input 18, thereby deploying the blade 12 and/or imager 14 of the probe apparatus 10.

Referring now to FIGS. 8B and 8C, the probe apparatus 10 is demonstrated in connection with the handle adapter 40 in an operating configuration 180. As shown in FIG. 8B, the trigger adapter 46 is shown at a resting position, where a trigger angle θ′ is shown extended away from the handle body 44 of the handle adapter 40. In FIG. 8C, the trigger adapter 46 is shown in a depressed configuration where the trigger angle θ′ is compressed toward the handle body 44. In response to the pressure applied to the input surface 174 of the trigger adapter 46, the change in the trigger angle θ′ may result in the engagement surface 162 being applied against the actuation lever 24. The force applied by the engagement surface 162 against the actuation input 18 of the actuation lever 24 may result in the lever angle θ changing, resulting in the deployment (e.g., translation, rotation, etc.) of the blade 12 and/or imager 14. Upon release of the input surface 174, the trigger adapter 46 may rotate about a pivotal trigger connection 182 connected to the adapter body 156 and return to the resting configuration demonstrated in FIG. 8B. As further demonstrated in FIGS. 8B and 8C, the rotation of the trigger adapter 46 about the pivotal connection 182 may result in the compression of the trigger angle θ′, resulting in the change in the lever angle θ, which further results in deployment of the blade 12 about the deployment angle ϕ. Additionally, as denoted in FIG. 8C, the probe body 16 may still be rotated relative to the main body 22 forming the handle portion 20 as denoted by the probe rotation R. In this configuration, the probe rotation R about the longitudinal axis A_{L} of the probe apparatus 10 may be adjusted to a desired probe angle ρ even with the probe apparatus 10 mounted to the handle adapter 40.

Referring now to FIG. 8D, the handle adapter 40 is demonstrated in an unloading configuration 190. The unloading configuration 190 may follow similar steps to those discussed in reference to FIG. 8B but reversed to withdraw the probe apparatus 10 from the recess 160. As shown, the unloading configuration 190 may be achieved by applying sequential steps including the following:
(D) rotating the latch 164 to disengage the locking protrusion 166;
(E) rotating the cover bracket 152 about the hinge assembly 154 exposing the receiving opening 158; and
(F) withdrawing the probe apparatus 10 from the recess 160 through the receiving opening 158.

By adjusting the handle adapter 40 to the unloading configuration 190, the probe apparatus 10 may easily be withdrawn and utilized with a cylindrical or fist-grip rather than the pistol-grip associated with the handle adapter 40. Additionally, as previously discussed, the probe apparatus 10 and handle adapter 40 may be selectively engaged and disengaged from the outer sheath 50a during a surgical procedure. Accordingly, the loading and unloading or mounting and demounting of the probe apparatus 10 to the handle adapter 40 may be completed by withdrawing the inner sheath 50b from the outer sheath 50a, thereby allowing the probe apparatus 10 to be implemented with the first or cylindrical-grip or the pistol-grip selectively during a surgical procedure without hardship or delay. In this way, the outer sheath 50a and, more generally, the configuration of the probe apparatus 10, including the plurality of nesting sheaths 50, may allow the probe apparatus to be flexibly implemented to suit a variety of user preferences and procedures without causing delays or complications that may impact patient outcomes.

As provided in various examples herein, the probe apparatus 10 may provide for several beneficial features that may be implemented to effectuate minimally invasive procedures. In particular, the plurality of nested sheaths 50 and corresponding operation of the imager apparatus 68 and blade 12 may provide for improved visibility and access to a targeted surgical site. The various details of the actuation assembly 30 as well as the handle adapter 40 may provide for and support flexible operation of the probe apparatus 10 without requiring additional specialty tools that may further complicate inventory and associated steps required for surgical procedures. Accordingly, the disclosure provides for a probe apparatus 10 as well as corresponding methods and systems to achieve minimally invasive surgical procedures while accommodating a wide variety of operating styles and techniques to suit user preferences.

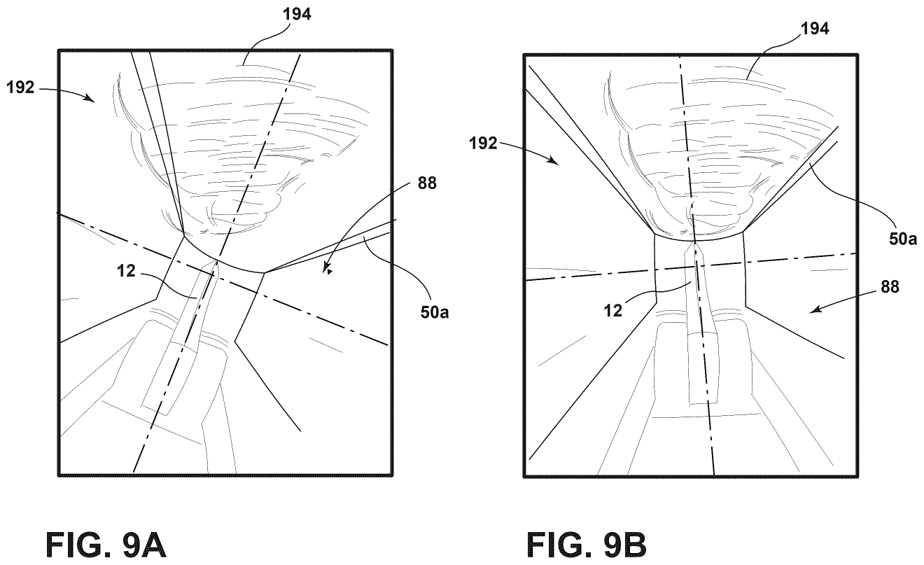

Referring now to FIGS. 9A and 9B, exemplary image data 192 of the field of view FOV captured by the imager 14, 138 is shown demonstrating the anatomy features of the probe apparatus 10 and patient anatomy 194. As shown, the image data 192 may depict the blade 12 positioned in the elongated slot 88 of the outer sheath 50a. The patient anatomy 194 or other visible features may be visible in the image data 192 through the outer sheath 50a. As previously described, the outer sheath 50a may be of a transparent material (e.g., transparent polymeric material), such that the field of view FOV of the imager 14, 138 may capture image data demonstrating the patient anatomy about the length of the outer sheath 50a as well as through the elongated slot 88. Such configurations may provide for improved visibility, allowing users to survey the patient anatomy on all sides of the outer sheath passage 52 while inserting and withdrawing the inner sheath 50b of the probe apparatus 10 from the outer sheath 50a.

In some implementations, the imager 14, 138 or, more generally, the imager apparatus 66 may include an inertial sensor (e.g., accelerometer, inertial measurement unit, gyroscope, etc.), which may supply orientation signals (e.g., gravitational direction, field directions, etc.) to a controller 202 of the imaging system 200. In this configuration, the controller (see FIG. 10) may track and offset the orientation of the field of view FOV of the image data 192 relative to gravity or another prevailing force in real time. The crosshairs demonstrated in FIGS. 9A and 9B demonstrate a change in orientation of the probe apparatus 10 and the imager 14, 138 relative to gravity. As the orientation of the anatomy 194 remains constant relative to gravity, the representation in the image data 192 also remains constant. However, the orientation of the probe varies relative to gravity and in correspondence with the crosshairs. In this way, the controller of the imaging system (FIG. 10) may adjust an orientation of the image data 192, such that the anatomy 194 or features depicted are consistently presented relative to gravity or a predefined offset relative to gravity regardless of the rotation of the probe apparatus 10 via the handle portion 22 or the probe rotation R of the rotational hub 32.

Recall, the actuation assembly 30 may provide for the probe rotation R via an engagement between the rotational hub 32 and the main body 22. As shown in FIGS. 9A and 9B, the orientation of the elongated slot 88 of the outer sheath 50a varies while the orientation of the patient anatomy 194 (e.g., a ligament) remains static or consistent in orientation. In the example shown, the appearance of the anatomy 194 varies slightly as a result of the interaction between outer sheath 50a of the probe 10 dilating or repositioning the tissue exemplifying the anatomy 194. However, the orientation of the anatomy 194 does not rotate as demonstrated by comparing the features of the elongated slot 88, the outer sheath 50a, and the blade 12 because the anatomy is not reoriented relative to gravity while the probe apparatus 10 is rotated. Accordingly, the controller of the imaging system may process the image data 192 responsive to the orientation of the imager 14, 138 to present the features of the operating environment consistently with respect to gravity or other prevailing/detectable forces or signals. For example, while gravity is mentioned specifically, other prevailing signals (e.g., artificial or natural magnetic fields), radio frequency signals, or similar signals may be detected to consistently orient the image data 192 relative to gravity or another direction of interest.

Referring now to FIG. 10, a block diagram of the imaging system 200 is shown. In various implementations, the system 200 may correspond to a video control console in communication with the imager 14, 138. The system 200 may further be in communication with various surgical tools via a controller 202. In addition to the imager 14, 138, the controller 202 may also be in communication with a surgical scope 204, which may correspond to various devices including an endoscope, laparoscope, arthroscope, etc. The imager 14, 138 may correspond to a chip-on-wire imaging device comprising the control circuitry incorporated in the imaging head 74 at the distal end portion of the imager apparatus 68. As shown, the scope 204 and the imager 14, 138 may be in communication with the controller 202 via a communication interface 206. Though shown connected via a conductive connection, the communication interface may correspond to a wireless communication interface operating via one or more wireless communication protocols (e.g., Wi-Fi, 802.11 b/g/n, etc.).

In various implementations, the imaging head 74 of the imager 14, 138 may incorporate one or more of the emitters 208, which may correspond to various light emitters configured to generate light in the visible range, the near infrared range, or various wavelengths. In various implementations, the emitters 208 may include light emitting diodes (LEDs), laser diodes, or other lighting technologies. The image sensor(s) 210 may correspond to various sensors and configurations comprising, for example, complementary metal-oxide semiconductor (CMOS) sensors, or similar sensor technologies. In some implementations, the imager 14, 138 may include an inertial sensor or directional sensor (e.g., accelerometer, inertial measurement unit, gyroscope, magnetometer etc.), which may supply orientation signals (e.g., gravitational direction, field directions, etc.) to the controller 202. In this configuration, the controller 202 may track and offset the orientation of the field of view FOV of the imager 14, 138 relative to gravity or another prevailing force in real time or with minimal delay associated with a framerate (e.g., 30 frames per second [FPS], 60 FPS, 90 FPS) of the image feed captured by the image sensor(s) 210. In this configuration a controller 202 may adjust an orientation of the image data 100, such that the anatomy 194 or features in the cavity are consistently presented relative to gravity or a predefined offset relative to gravity regardless of the rotation of the imager 14, 138.

In various implementations, the imager 14, 138 may comprise the control circuitry configured to control the operation of image sensor(s) 210 and the emitter(s) 208 as well as process and/or communicate the image data 192 to the controller 202. Additionally, the control circuitry may be in communication with a user interface 214, which may include one or more input devices, indicators, displays, etc. The user interface 214 may provide for the control of the imager 14, 138 including the activation of one or more control routines. The user interface 214 may provide for the selection, adjustment, or toggling of one or more of the image feeds associated with the operation of the imager 14, 138 and/or the scope 204. The control circuitry may be implemented by various forms of controllers, microcontrollers, application-specific integrated controllers (ASICs), and/or various control circuits or combinations.

The controller 202 or system controller may comprise a processor 216 and a memory 218. The processor 216 may include one or more digital processing devices including, for example, a central processing unit (CPU) with one or more processing cores, a graphics processing unit (GPU), digital signal processors (DSPs), field programmable gate arrays (FPGAs), application-specific integrated circuits (ASICs) and the like. In some configurations multiple processing devices are combined into a System-on-a-Chip (SoC) configuration while in other configurations the processing devices may correspond to discrete components. In operation, the processor 216 executes program instructions stored in the memory 218 to perform various operations related to the operation of the imaging system 200 as well as one or more surgical control consoles 222 in communication with the controller 202.

The memory 218 may comprise one or more data storage devices including, for example, magnetic or solid-state drives and random access memory (RAM) devices that store data. The memory 218 may include one or more stored program instructions, object detection templates, image processing algorithms, etc. In various implementations, the controller 202 may correspond to a display or video controller configured to output formatted image data to one or more display devices 220. In such applications, the controller 202 may include one or more formatting circuits 224, which may process the image data received from the imager 14, 138 and/or the surgical scope 202, communicate with the processor 216, and process the image data for presentation on the one or more display devices 220. The formatting circuits 224 may include one or more signal processing circuits, analog-to-digital converters, digital-to-analog converters, etc. The user interface 214 of the controller 202 may be in the form of an integrated interface (e.g., a touchscreen, input buttons, an electronic display, etc.) or may be implemented by one or more connected input devices (e.g., a tablet) or peripheral devices (e.g., keyboard, mouse, foot pedal, etc.).

As shown, the controller 202 may also be in communication with an external device or server 230, which may correspond to a network, local or cloud-based server, device hub, central controller, or various devices that may be in communication with the controller 202 and, more generally, the imaging system 200 via one or more wired (e.g., serial, Universal Serial Bus (USB), Universal Asynchronous Receiver/Transmitter (UART), etc.) and/or wireless communication interfaces (e.g., a ZigBee, an Ultra-Wide Band (UWB), Radio Frequency Identification (RFID), infrared, Bluetooth®, Bluetooth® Low Energy (BLE), Near Field Communication (NFC), etc.) or similar communication standards or methods. For example, the controller 202 may receive updates to the various modules and routines as well as communicate sample image data from the imager 14, 138 to a remote server for improved operation, diagnostics, and updates to the imaging system 200. The user interface 214, the external server 230, and/or a surgical control console may be in communication with the controller 202 via one or more I/O circuits 232. The I/O circuits 232 may support various communication protocols including, but not limited to, Ethernet/IP, TCP/IP, Universal Serial Bus, Profibus, Profinet, Modbus, serial communications, etc.

According to some aspects of the disclosure, a probe apparatus for selectively cutting tissue comprises a plurality of nested sheaths including an inner sheath forming an inner sheath passage and an outer sheath forming an outer sheath passage. A cannula forming lumen and an actuator linkage extend through an inner sheath passage from a main body comprising a handle portion at a proximal linkage end portion to a distal linkage end portion. A blade is in connection with the distal linkage end portion and the inner sheath, while an imaging apparatus comprising an imager extends through the lumen and is configured to capture image data in a field of view directed from the distal linkage end portion.

According to various aspects, the disclosure may implement one or more of the following features or configurations in various combinations:
the cannula forms a distal opening through the distal linkage end portion, wherein the imager protrudes at a projection angle relative to the distal end portion of the actuator linkage;
the distal opening is angled through a side wall of the cannula forming the projection angle of the imager;
a path of the lumen is aligned with a cutting edge of the blade along the projection angle;
the actuator linkage extends along a linkage axis through the inner sheath passage and the cannula forms a sloped passage angled relative to the linkage axis proximal to the linkage end portion;
the outer sheath is selectively attached to the probe apparatus slidably engaging the inner sheath and rotationally engaged to a rotational hub in connection the main body;
a rotational hub forming a coupling interface rotationally engaged with a proximal outer sheath portion of the outer sheath and in connection with the main body, wherein the rotational hub rotates about the longitudinal body axis of the main body;
an actuator assembly comprising an actuation lever in connection with the main body;
the actuation lever pivotally engages the main body and the actuator linkage translates responsively adjusting a blade position of the blade;
the actuator assembly further comprises a longitudinal actuator in connection with the rotational hub and the actuation linkage within the main body;
the longitudinal actuator forms an interior imager passage extending through the rotational hub and in connection with the cannula;
the imager passage receives a coupling interface formed by a portion of the imaging apparatus along a communication interface;
the longitudinal actuator translates within a longitudinal aperture formed in the rotational hub in response to an actuation of the actuation lever;
the longitudinal aperture receives a distal protrusion of the longitudinal actuator and a mating engagement between the longitudinal aperture and the distal protrusion rotationally couples the longitudinal actuator to the rotational hub;
the mating engagement is formed by an irregular profile shape of the distal protrusion about the longitudinal axis;
the distal protrusion and the longitudinal aperture are noncircular;
the longitudinal actuator connects to the main body via the rotational hub and is rotationally coupled to the rotational hub;
the actuation lever comprises at least one engagement arm extending between actuation surfaces longitudinally spaced along a length of the longitudinal actuator;
a change in a position of the actuation lever causes the at least one engagement arm to engage one of the actuation surfaces and the longitudinal actuator responsively translates;
the longitudinal actuator is free to rotate about the longitudinal axis relative to the engagement arm;
at least one of the plurality of nested sheaths forms a curved engagement profile extending along a least a portion of an engagement length that is inserted into the patient anatomy;
the curved engagement profile comprises a radial curve having a radius greater than 50 cm, 100 cm, 150 cm, or 200 cm; and/or
the curved engagement profile is between approximately 50 cm and 300 cm.

According to another aspect of the disclosure, a handle adapter apparatus for a surgical probe comprises a closure comprising a base and a closure bracket forming a receiving opening that forms an elongated profile shape complementary to a main body of the surgical probe. A handle body extends from the base substantially transverse to the receiving opening. A trigger adapter comprises an input surface and an engagement surface, with the trigger adapter pivotally engaged with the base and extending from the handle body. The engagement surface of the actuator aligns with an actuation lever of the surgical probe positioned within the receiving opening.

According to various aspects, the disclosure may implement one or more of the following features or configurations in various combinations:
the trigger adapter rotates about a pivotal connection with the base and engages the actuation lever in response to an angular displacement of the input surface about the pivotal connection;
the input surface is substantially perpendicular to the engagement surface;
the closure bracket is pivotally connected to the base along a length of the profile shape;
the closure comprises a latch that selectively locks the closure bracket to the base, securing the main body within the receiving opening;
the actuation lever is in connection with the main body of the surgical probe and configured to selectively actuate a distal actuator of the surgical probe;
the base forms an actuator opening extending between the receiving opening and the engagement surface, wherein the actuator opening receives the actuation lever within the receiving opening; and/or
the elongated profile shape comprises a cylindrical profile.

According to yet another aspect of the disclosure, a method for deploying a surgical probe comprises inserting a dilator sheath into a patient anatomy via an access aperture; inserting a blade sheath within a dilator sheath passage from a proximal dilator sheath end portion to a distal dilator sheath end portion; and sliding an actuator linkage through a blade sheath passage extending through the blade sheath, thereby translating a position of a field of view of an imager and a cutting angle of a blade proximal to a distal linkage end portion.

According to various aspects, the disclosure may implement one or more of the following features or configurations in various combinations:
the insertion of the dilator sheath forms an interior passage through the patient anatomy to a target region;
the target region comprises a transverse carpal ligament;
the imager captures image data demonstrating the patient anatomy between the proximal dilator sheath end portion to a distal dilator sheath end portion contemporaneous to the insertion of the blade sheath within the dilator sheath passage;
the image data is captured through an elongated slot form along a length of the dilator sheath and the field of view is aligned with a cutting edge of the blade;
inserting the imager into a cannula extending through the actuator linkage; and/or
the access aperture comprises a transverse incision proximate to a wrist flexion crease.

According to some aspects of the disclosure, an imaging apparatus for a probe apparatus for selectively cutting tissue includes a handle portion forming a main body extending along a longitudinal axis and at least one sheath forming a sheath passage in connection with the main body. A cutting tool is operably coupled to the handle portion and positioned at a distal end portion of the at least one sheath. An imaging apparatus includes an imager and a directional sensor, wherein the imaging apparatus is positioned in the distal end portion of the sheath and captures image data in a field of view directed toward the cutting tool, wherein the image data depicts a local environment and at least an observed portion of the probe apparatus. A controller is in communication with the imager and the inertial senor, wherein the controller receives the image data and orientation data from the directional sensor, and in response to the orientation data, offsets an orientation of a depiction of the local environment while displaying an updated relative position of the observed portion of the probe apparatus.

According to various aspects, the disclosure may implement one or more of the following features or configurations in various combinations:
the observed portion of the probe apparatus comprises the cutting tool;
the local environment comprises an anatomy of a patient;
the sheath is of an at least partially transparent material and the anatomy is visible in the image data through the at least partially transparent material;
the anatomy is accessed within a patient cavity;
the cutting tool comprises a cutting blade in connection with an actuator in the handle portion, wherein the blade is selectively deployed over a deployment range in response to an actuation of the actuator; and/or
the blade is depicted in the field of view over the deployment range.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

The claims:

1. A probe apparatus for selectively cutting tissue, the probe apparatus comprising:
a handle portion forming a main body extending along a longitudinal axis;
a plurality of nested sheaths comprising an inner sheath forming an inner sheath passage and an outer sheath forming an outer sheath passage in connection with the main body;
a cannula forming lumen and an actuator linkage extending through the inner sheath passage from at a proximal linkage end portion to a distal linkage end portion;
a blade in connection with the distal linkage end portion and the inner sheath; and
an imaging apparatus comprising an imager, wherein the imaging apparatus extends through the lumen and is configured to capture image data in a field of view directed from the distal linkage end portion.

2. The probe apparatus according to claim 1, wherein the cannula forms a distal opening through the distal linkage end portion, wherein the imager protrudes at a projection angle relative to the distal linkage end portion.

3. The probe apparatus according to claim 2, wherein the distal opening is angled through a side wall of the cannula defining the projection angle of the imager.

4. The probe apparatus according to claim 3, wherein a path of the lumen is aligned with a cutting edge of the blade along the projection angle.

5. The probe apparatus according to claim 1, wherein the actuator linkage extends through the inner sheath passage and the cannula forms a sloped passage angled outward from the inner sheath and the outer sheath.

6. The probe apparatus according to claim 1, wherein the outer sheath is selectively attached to the probe apparatus slidably engaging the inner sheath, and wherein the outer sheath is rotationally engaged to a rotational hub in connection with the main body.

7. The probe apparatus according to claim 1, further comprising a rotational hub forming a coupling interface rotationally engaged with a proximal outer sheath portion of the outer sheath and in connection with the main body, wherein the rotational hub rotates about the longitudinal axis of the main body.

8. The probe apparatus according to claim 7, further comprising an actuator assembly comprising an actuation lever in connection with the main body.

9. The probe apparatus according to claim 8, wherein the actuation lever pivotally engages the main body and the actuator linkage translates responsively adjusting a blade position of the blade.

10. The probe apparatus according to claim 8, wherein the actuator assembly further comprises:
a longitudinal actuator in connection with the rotational hub and the actuation linkage within the main body, wherein the longitudinal actuator is spaced from the rotational hub via a biasing spring.

11. The probe apparatus according to claim 10, wherein the longitudinal actuator forms an interior imager passage extending through the rotational hub and in connection with the cannula.

12. The probe apparatus according to claim 11, wherein the imager passage receives a coupling interface formed by a portion of the imaging apparatus along a communication interface.

13. The probe apparatus according to claim 10, wherein the longitudinal actuator translates within a longitudinal aperture formed in the rotational hub in response to an actuation of the actuation lever.

14. A method for deploying a surgical probe, the method comprising:
inserting a dilator sheath into a patient anatomy via an access aperture;
inserting a blade sheath within a dilator sheath passage from a proximal dilator sheath end portion to a distal dilator sheath end portion; and
sliding an actuator linkage through a blade sheath passage extending through the blade sheath, thereby translating a position of a field of view of an imager and a cutting angle of a blade proximal to a distal linkage end portion.

15. The method according to claim 14, wherein the insertion of the dilator sheath forms an interior passage through the patient anatomy to a target region comprising a transverse carpal ligament.

16. The method according to claim 14, wherein the imager captures image data demonstrating the patient anatomy between the proximal dilator sheath end portion and a distal dilator sheath end portion contemporaneous to the insertion of the blade sheath within the dilator sheath passage.

17. The method according to claim 16, wherein the image data is captured through an elongated slot formed along a length of the dilator sheath and the field of view is aligned with a cutting edge of the blade.

18. The method according to claim 14, further comprising:
inserting the imager into a cannula extending through the actuator linkage, and, wherein the access aperture comprises a transverse incision proximate to a wrist flexion crease.

19. An imaging apparatus for a probe apparatus for selectively cutting tissue, the probe apparatus comprising:
a handle portion forming a main body extending along a longitudinal axis;
at least one sheath forming a sheath passage in connection with the main body;
a cutting tool operably coupled to the handle portion and positioned at a distal end portion of the at least one sheath;
an imaging apparatus comprising an imager and a directional sensor, wherein the imaging apparatus is positioned in the distal end portion of the sheath and captures image data in a field of view directed toward the cutting tool, wherein the image data depicts a local environment and at least an observed portion of the probe apparatus; and
a controller in communication with the imager and the inertial senor, wherein the controller receives the image data and orientation data from the directional sensor, and in response to the orientation data, offsets an orientation of a depiction of the local environment while displaying an updated relative position of the observed portion.

20. The imaging apparatus according to claim 19, wherein the observed portion of the probe apparatus comprises the cutting tool and the sheath is of an at least partially transparent material and the anatomy is visible in the image data through the at least partially transparent material.
